(19) United States
(12) Patent Application Publication
(10) Pub. No.: US 2024/0390235 A1
(43) Pub. Date: Nov. 28, 2024

(54) APPARATUS AND METHOD FOR NASOGASTRIC TUBE INSERTION GUIDE

(71) Applicant: E.Z.N.G TECHNOLOGIES LTD, Moshav Bizaron (IL)

(72) Inventors: Josef OSTERWEIL, Rockville, MD (US); Zvi M OSTERWEIL, New York, NY (US)

(21) Appl. No.: 17/915,130

(22) PCT Filed: Mar. 26, 2021
(86) PCT No.: PCT/US2021/024309

§ 371 Date: May 16, 2024

Related U.S. Application Data
(60) Provisional application No. 63/100,763, filed on Mar. 30, 2020.

(51) Int. Cl. A61J 15/00; A61B 90/00; A61M 25/01

(52) U.S. Cl. CPC A61J 15/0084; A61B 90/06; A61J 15/0003; A61B 2090/064; A61B 2562/12; A61M 2025/0166

(57) ABSTRACT

An apparatus for identifying the location of a nasogastric tube that includes a pump that selectively fluidly couples to a nasogastric tube, a pressure sensor that identifies the pressure of an inner chamber of the nasogastric tube, and a controller configured to selectively power the pump and read the pressure sensor to identify an impedance of at least one opening of the nasogastric tube.

### CROSS-REFERENCE TO RELATED APPLICATION

The present disclosure claims the benefit of U.S. Provisional Application No. 63/100,763 filed Mar. 30, 2020 and titled “APPARATUS AND METHOD FOR NASOGASTRIC TUBE INSERTION GUIDE”, the contents of which are hereby incorporated herein in entirety.

### FIELD OF THE DISCLOSURE

The present disclosure relates to the proper insertion of a nasogastric tube and more specifically to an apparatus and method for directing the proper insertion of the nasogastric tube by producing and monitoring a pressure therein.

### BACKGROUND

The present disclosure relates to the insertion of medical tubes, such as, but not limited to, nasogastric feeding tubes, and particularly their proper entry into the stomach through the esophagus. Patients who cannot be fed in conventional ways may require a nasogastric tube (hereinafter “NGT”) as a conveyance of nutrition into the stomach. Often, insertion of the NGT is attempted without utilizing advanced imaging systems such as X-Ray machines and the like during the insertion. Rather, verification that the distal end of the tube was properly positioned in the stomach is done after the NGT is supposedly positioned therein. The conventional verification techniques are: aspiration of gastric content and pH measurement; X-Ray tracking; auscultation while forcing a small amount of air down the tube; and measuring pH in the distal end of the tube while in the stomach, among others. These techniques are often messy, expensive, dangerous (i.e., through radiation exposure during X-Ray), and inconclusive.

Insertion of a nasogastric tube incorrectly can lead to injury and cause food contamination of the respiratory system. Consequently, it is desired to provide the caregiver with guidance so the NGT enters into the esophagus on the way to the stomach and not into the trachea.

### SUMMARY

The instant disclosure applies the principles of gas and fluid dynamics and anatomical structures. These principles are utilized in guiding a NGT into the stomach.

One objective of the instant disclosure is to ensure that the NGT is inserted into the esophagus on the way to the stomach early in the insertion process. Early identification of esophagus entry may prevent the NGT from substantially entering the trachea and the lungs, thereby preventing the damage associated with improper placement of the NGT. A patient, human or animal, must be fed using the NGT when conventional ways of obtaining nourishment are not possible. The caregiver may find the use of the NGT is the preferred option to provide nourishment to the patient. The NGT must be inserted safely and efficiently. If the tube is advanced into the trachea instead of into the esophagus, damage can occur while the tube is being inserted and food contamination can cause more critical damage to the respiratory system.

Another objective of the present disclosure is determining early in the procedure if the NGT is being advanced into the esophagus or into the trachea. Thus, if the NGT is advancing into the trachea, it can be withdrawn and the procedure restarted so the NGT is properly guided into the esophagus to thereby avoid causing damage to the respiratory system and neighboring tissue. Nourishment is deposited through the proximal end of the NGT and released into the stomach through an eye-like opening on the side at the distal end of the NGT. It is therefore necessary to guide the NGT through the esophagus into the stomach.

The present disclosure considers the anatomically distinct features of the esophagus and the trachea, both accessible from the nasal cavity. The esophagus is a muscular tube connecting the pharynx to the stomach. The tubular structure of the esophagus is normally in a collapsed state until it is dilated by nourishment intake or other external means. The trachea is a large membranous tube reinforced by cartilage rings. It connects the larynx to the bronchial tubes and down into the lungs. The trachea is an open structure so as not to impede breathing.

A NGT has an eye-like opening on the side of the distal end of the tube. When inadvertently placed in the trachea, it is loosely surrounded by the trachea and airflow through the NGT is not impeded in either flow direction. A feature of the present disclosure is that when inserted into the esophagus, the tube is surrounded by the inner wall tissue of the esophagus, thus impeding air flow in both directions.

In one aspect of the present disclosure the use of the difference in airflow impedance values when the tube is in the trachea versus the esophagus is a consideration during NGT insertion guidance. Furthermore, once the NGT reaches the stomach, air flow impedance is slightly lower than it is in the esophagus and can be used to confirm entry into the stomach. If the NGT is further advanced into the duodenum (i.e., small intestine), the impedance to air flow may further change.

Another aspect of this disclosure is detection of a kinked NGT which can occur during tube insertion. A kinked NGT has a reduced effective length i.e. air volume trapped between the location of the kink and the apparatus. This reduced air volume will further increase the measured airflow impedance relative to airflow impedance when the tube is in the esophagus. One aspect of this disclosure includes marking the NGT at the length from the nose to the ear and then to the stomach. During insertion, the markings may be considered in identifying the positioning of the NGT.

In another aspect of this disclosure, when the stomach mark reaches the nose after traversing the esophagus, a decrease in airflow impedance is expected. Lack of change in airflow impedance is an indication that the NGT is kinked. Similarly, the NGT may be marked to indicate when it reaches the low neck point. When this mark reaches the nose during NGT insertion, the NGT is either in the esophagus or in the trachea, so attention must be focused on the difference in airflow impedance.

In another aspect of this disclosure, a calibration is performed with the eyes of the tube obstructed mimicking the condition in the esophagus. In yet another aspect of the present disclosure a quick calibration process occurs for each individual NGT thus overcoming the difference in tube parameters while at the same time compensating for loose apparatus components and assembly tolerances.

In another aspect of the present disclosure the implementation of a ‘Whoosh Test’ where air is injected into the tube while auscultating the stomach area. As part of this test, a whoosh sound confirms that the tube is in the stomach.

Another aspect of the present disclosure is the sensing of air flow impedance in the NGT by repeated momentary measurements so as to minimize internal friction while the tube is being advanced. The measurement can be implemented in a sequential manner but other measurement patterns do not deviate from the scope of the current disclosure.

Another option of the present disclosure is air flow impedance monitoring in aspiration mode. In the aspiration mode, the impedance differences are more pronounced.

In yet another aspect of the present disclosure a determination of air flow based on the pressure in the NGT for each momentary air flow sensing event is executed.

Another aspect of the present disclosure involves keeping the pressure buildup low by letting the pressure reach a predetermined level and recording the time it took to reach this level. In this way, pressure does not exceed a predetermined level before airflow impedance measurement (aspiration and injection) stops.

In another aspect of this disclosure a digital and/or a graphic curve display of the time it takes to reach the predetermined pressure for each measurement may be implemented. Further, a light and/or audio indicator may be used instead of or in addition to the display.

Yet another aspect of the present disclosure is verification of the hydrochloric acid in the stomach using a thin probe that is inserted into the lumen of the tube. The chemical reaction of the material at the tip of the probe with the gastric fluids changes the appearance of the probe tip. This serves as a confirmation that the distal end of the tube is inside the stomach.

Another aspect of the present disclosure is that the chemicals used with the probe and their derivative compounds are not toxic in the human digestive tract.

Another aspect of the present disclosure is the stiffening of the tube by the probe while inside the lumen, thus providing an advantage in advancing the tube.

In yet another aspect of the present disclosure an optional protective filter is positioned between the NGT and the apparatus.

One embodiment of the present disclosure is an apparatus for identifying the location of a nasogastric tube. The apparatus may have a pump configured to be selectively fluidly coupled to a nasogastric tube, a pressure sensor configured to identify the pressure of an inner chamber of the nasogastric tube, and a controller configured to selectively power the pump and read the pressure sensor to identify an impedance of at least one opening of the nasogastric tube.

One example of this embodiment has a filter positioned to filter fluid passing between the pump and the nasogastric tube. Another example has a pressure release configured to selectively fluidly couple the inner chamber of the nasogastric tube to a surrounding environment.

In yet another example of this embodiment, the controller has a pressure threshold stored therein. In one aspect of this example, the controller has a calibration process stored therein and the controller selectively executes the calibration process to determine a time threshold for generating the pressure threshold.

In another example, the pump runs intermittently. In another example, the controller is configured to determine airflow impedance based on a pressure time factor. In yet another example, the controller is configured to stop the pump when the pressure sensor identifies that a predetermined pressure is reached. In yet another example, the controller is configured to determine the air flow impedance based on the time it takes the pump to generate a threshold pressure in the inner chamber.

Another embodiment of this disclosure is a method for manufacturing an apparatus for identifying the location of a nasogastric tube. The method includes positioning a pump to selectively move fluid through a coupler configured be selectively fluidly coupled to a nasogastric tube. The pump is configured to selectively move fluid to or from an inner chamber of the nasogastric tube when fluidly coupled thereto. The method also includes fluidly coupling a pressure sensor between the pump and the coupler to selectively identify a pressure of the inner chamber when coupled to the nasogastric tube. Another part of this method is communicatively coupling a controller to the pump and pressure sensor to provide instructions to selectively power the pump and selectively identify the pressure of the inner chamber. This method also considers programming the controller to selectively power the pump and measure the pressure of the inner chamber when fluidly coupled to the nasogastric tube to identify an impedance of an opening of the nasogastric tube.

One example of this embodiment includes programming the controller to discontinue powering the pump if the pressure of the inner chamber does not meet a pressure threshold after a predetermined amount of time powering the pump. Part of this example includes programming a calibration process in the controller to establish a time threshold for a specific nasogastric tube. Another part of this example includes programming the controller to store the time threshold established during the calibration and use the time threshold as the predetermined amount of time.

Another example of this embodiment includes providing a user input in communication with the controller and configured to be engaged by the user when the nasogastric tube is at a predefined location in a patient. Part of this example includes programming the controller to power the pump and monitor the pressure sensor when the user input is engaged.

Yet another example of this embodiment includes programming the controller to communicate with a stop button wherein the controller is configured to not power the pump when an input from the stop button is identified.

Yet another embodiment of this disclosure is a method of identifying the positioning of a nasogastric tube in the stomach of a patient. The method includes fluidly coupling a nasogastric tube to a pump, selectively powering the pump to alter the pressure in an inner chamber of the nasogastric tube, monitoring the pressure in the inner chamber while the pump is powered and identifying the location of an opening of the nasogastric tube by determining the impedance of the opening.

In one example of this embodiment, the impedance is determined by monitoring the time interval that the pump is powered and the pressure in the inner chamber. In another example the impedance is determined at least twice while inserting the nasogastric tube into the stomach of the patient.

### BRIEF DESCRIPTION OF THE DRAWINGS

The above-mentioned aspects of the present disclosure and the manner of obtaining them will become more apparent and the disclosure itself will be better understood by reference to the following description of the embodiments of the disclosure, taken in conjunction with the accompanying drawings, wherein:

FIG. 1 is a drawing showing cross-sections of the trachea and the esophagus;

FIG. 2 is a drawing showing a NGT connected to the NGT insertion guide via a protective filter, according to the present disclosure;

FIG. 3a is a drawing showing the detailed block diagram of the NGT insertion guide of FIG. 2

FIG. 3b is another detailed block diagram of an embodiment of this disclosure;

FIG. 4 is a drawing of the distal end of the NGT having an eye-like opening for nourishment delivery into the stomach;

FIG. 5 is a drawing of the probe used to identify hydrochloric acid of the stomach;

FIGS. 6a-6c are logic flow charts of one embodiment of this disclosure;

FIG. 7 is a block diagram of a manufacturing method of this disclosure;

Corresponding reference numerals are used to indicate corresponding parts throughout the several views.

### DETAILED DESCRIPTION

For the purposes of promoting an understanding of the principles of the present disclosure, reference will now be made to the embodiments described herein and illustrated in the drawings and specific language will be used to describe the same. It will nevertheless be understood that no limitation of the scope of the present disclosure is thereby intended, such alterations and further modifications in the illustrated devices and methods, and such further applications of the principles of the present disclosure as illustrated therein being contemplated as would normally occur to one skilled in the art to which the present disclosure relates.

The particulars shown herein are by way of example and for purposes of illustrative discussion of embodiments of the present disclosure only, and are presented in the cause of providing what is believed to be the most useful and readily understood description of the principles and conceptual aspects of the present disclosure. In this regard, no attempt is made to show details of the present disclosure in more detail than is necessary for the fundamental understanding of the present disclosure, the description taken with the drawings making apparent to those skilled in the art how the present disclosure may be embodied in practice.

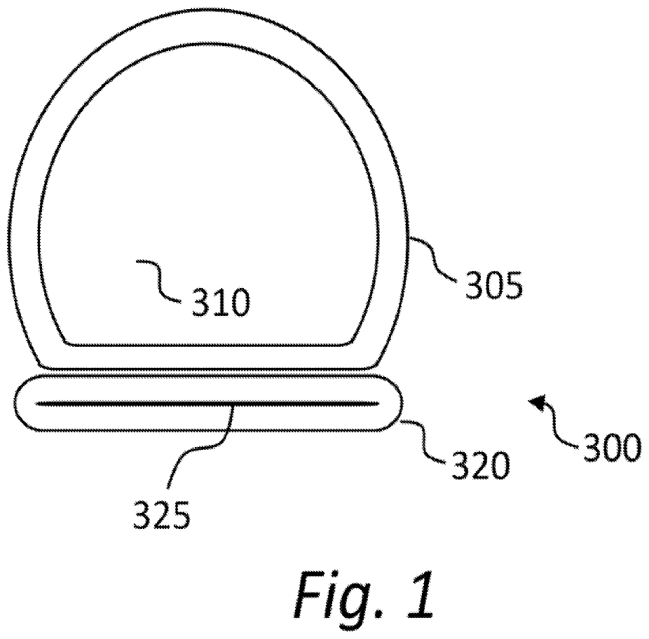

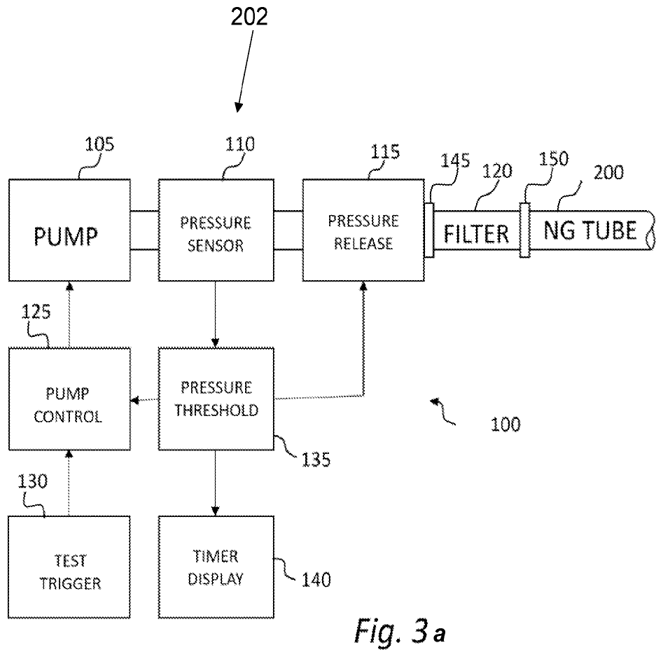

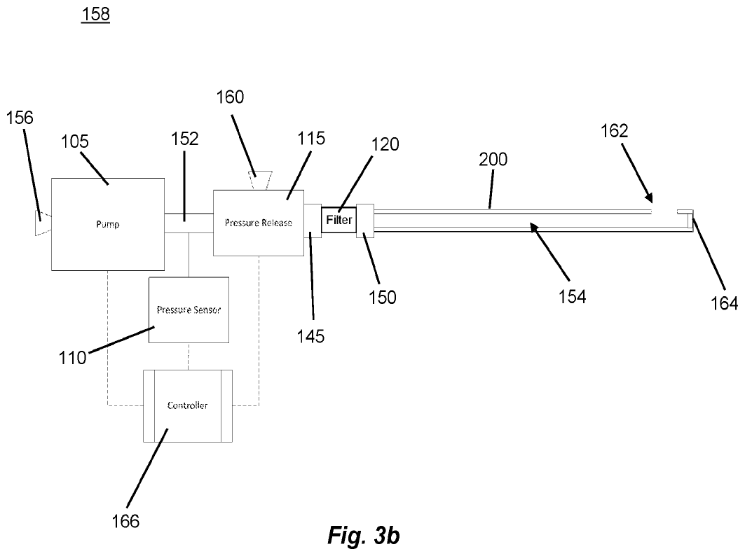

FIG. 1 illustrates the cross-section of the trachea and the esophagus 300. The trachea 305 walls are reinforced with cartilage rings that keep the lumen 310 of the trachea extended and open to unimpeded air flow, so as to enable breathing. When a nasogastric tube or NGT 200 (see FIG. 3a) is inserted into the trachea 305, air flow in the NGT 200 is not impeded in either direction.

The esophagus 320 whose lumen 325 is normally collapsed, typically at least partially impedes air flow. The lumen 325 can be dilated by, but not limited to, food/drink, injection of air and medical instruments among other reasons. When a NGT is inserted into the esophagus 320, the lumen 325 surrounds the NGT, at least partially blocking the NGT's distal end eye-like opening 162 (see FIG. 3b). Thus, air flow in the NGT 200 is typically at least partially impeded when the opening 162 of the NGT 200 is positioned in the esophagus 320. This disclosure contemplates identifying the impedance of air into the opening 162 of the NGT 200 as an indication that the distal end 164 of the NGT 200 is in the gastric tract (i.e. esophagus 320) and may be advanced into the stomach among other things.

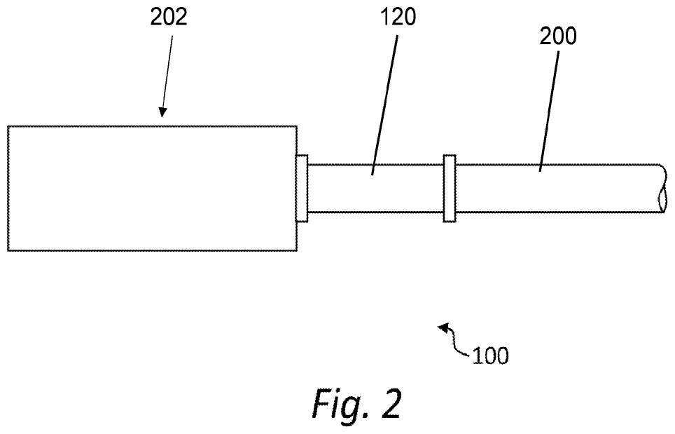

FIG. 2 illustrates a top level assembly 100 of the NGT insertion guide 202, hereafter referred to as “apparatus.” The apparatus 202 is connected to the NGT 200 through a filter 120. In one aspect of this disclosure, the apparatus 202 measures the air flow impedance in the NGT 200 among other things.

FIG. 3a illustrates a detailed functional block diagram of the NGT insertion guide 202 including an optional filter 120 coupled to the NGT 200. The filter 120 provides protection against contamination from one patient to the next among other things. The filter 120 is defined herein as a passive filter such as a membrane or an active filter that physically separates the tube from the apparatus 202 when the flowing air could contain contaminants, pathogens, etc. This functional block diagram illustrates one configuration as an example but other configurations and connectivity patterns do not deviate from the scope of the current disclosure. More specifically, this disclosure contemplates utilizing any known filtration method to separate the NGT 200 from the apparatus 202 to avoid potential cross-contamination between patients. Further still, in another embodiment considered herein there may not be a filter 120 at all.

A pressure source such as, but not limited to, a pump 105 may selectively deliver airflow to the NGT 200. The pump 105 is controlled by pump control 125. Further, a trigger 130 may selectively initiate the apparatus 202 through the pump control 125 and pump 105. Airflow in the NGT 200 is initiated and airflow impedance is measured by a combination of identifying the pressure in the NGT 200 and the pump 105 action time (i.e., pumped air volume). In other words, this disclosure considers identifying the pressure time factor (i.e., air pressure and volume) of the NGT 200 to identify the relative position of the distal end of the NGT 200 in the patient.

FIG. 3b illustrates another block diagram of the present disclosure. FIG. 3b illustrates the pump 105 fluidly coupled to a pressure release 115 via conduit 152. However, the pressure release 115 may be directly coupled to the pump 105 as well. Regardless, the pressure release 115 may be coupled to the filter 120 through a filter coupler 145, or directly coupled to the NGT 200 through an NGT coupler 150. Further still, other embodiments considered herein may not have a pressure release at all and the NGT 200 may be directly coupled to the conduit 152 through the NGT coupler 150. The NGT 200 may be coupled to the filter 120 (or directly to the pressure release 115 or the conduit 152 in alternative embodiments) through the NGT coupler 150 so an inner chamber 154 of the NGT 200 is fluidly coupled to the filter 120, pressure release 115, conduit 152, and pump 105 as in the exemplary embodiment of FIG. 3b. In this configuration, the pump 105 may pump fluid into, or out of, the inner chamber 154 through the conduit 152, pressure release 115, and filter 120.

The pump 105 may have a pump orifice 156 that provides a fluid input or exhaust for the pump 105 to a surrounding environment or atmosphere 158. When the pump 105 is adding fluid to the inner chamber 154, the pump orifice may draw air, or other fluid, out of the surrounding environment 158 and into the inner chamber 154. Further, in another embodiment the pump 105 may draw fluid out of the inner chamber 154 and exhaust the fluid into the surrounding environment 158. Similarly, the pressure release 115 may have a release orifice 160 that allows the pressure release 115 to selectively fluidly couple the inner chamber 154 to the surrounding environment 158.

The NGT 200 may be substantially isolated from the surrounding environment 158 except through one or more opening 162 at a distal end of the NGT 200. In a typical NGT 200, the opening 162 is intended to be positioned within the stomach to direct nutrients thereto.

A controller 166 may communicate with and/or control one or more of the components of the apparatus 202. More specifically, the controller may selectively power the pump 105 to move fluid into, or out of, the inner chamber 154. Further, the controller 166 may communicate with the pressure sensor 110 to identify the pressure of the fluid in the inner chamber 154. Similarly, the controller 166 may control the pressure release to selectively fluidly couple the inner chamber 154 to the surrounding environment 158 among other things. Alternatively, the pressure release 115 may be preset to release pressure when the inner chamber 154 generates a pressure outside of a preset pressure release pressure.

Airflow impedance can be measured by letting the pressure reach a predetermined value and determining the time it took for the pressure to reach this level. A shorter time corresponds to higher impedance. Alternately, the pump 105 may be pulsed with a constant pulse duration, and the built-up pressure may correspond to air flow impedance in the NGT 200. More specifically, higher pressure readings may correspond to higher air flow impedance. The air flow impedance may be determined by the following formula:

$$A = P / T \qquad Formula I$$

In Formula I, air flow impedance is A, pressure is P, and time is T. In Formula I, time T represents the air volume V pumped at a constant pumping rate. A pressure threshold and a time (volume) threshold are two opposite alternatives. However, a combination pattern in between the pressure and time threshold is also contemplated herein and within the scope and spirit of the present disclosure. In one aspect of this disclosure, the pump 105 may be a piston-based pump and the pressure P may be linearly derived from the driving force F of the pump. In this example, the driving force F threshold could substitute P threshold from Formula I.

In one example, when the pressure in the NGT 200 reaches a predetermined level of pressure, a pressure threshold 135 is met and the pump 105 is stopped by the pump control 125. At the same time, the apparatus 202 may record the time that it took the pump 105 to reach the threshold pressure 135 on a timer display 140 or otherwise store the recorded time on a memory unit for further processing. The time indicated on the timer display 140 or otherwise stored in the apparatus 202 corresponds to the level of air flow impedance. If the pressure threshold 135 is not identified by monitoring the pressure sensor 110 within a predetermined interval or cutoff time, the pressure threshold 135 may communicate with the pump control 125 to stop the pump 105. At the same time, the apparatus 202 may identify the time (numerically, graphically, by audio or lights) that it took to have the pump 105 stopped. In one example, the time may be displayed on the timer display 140. The time indicated on the timer display 140 may correspond to the level of air flow impedance and in the present example may be representative of low impedance.

In one application of the present disclosure, impedance will be very low when the opening 162 of the NGT 200 is positioned in the nasal cavity of the user. However, by the time the opening 162 of the NGT 200 is advanced approximately to the level of the middle of the user's neck, the distal end of the NGT 200 will enter either the esophagus 325 or the trachea 310 when advanced further. If the timer display 140 indicates high air flow impedance when the NGT 200 is in this position, it is evidence that the opening 162 of the NGT 200 has entered the lumen of the esophagus 325 and advancement into the stomach can proceed. Once in the stomach, a momentary drop in air flow impedance may further confirm the location of the opening 162 of the NGT 200. However, if the air flow impedance does not increase as the NGT 200 is advanced farther down the user's neck, the opening 162 at the distal end of the NGT 200 is likely in the trachea 310. This is an indication that the healthcare provider should pull the NGT 200 back to avoid improperly positioning the NGT 200 at least partially in the lungs of the user.

A kinked NGT 200 can occur during the tube insertion process. If the NGT 200 becomes kinked during insertion, the distal end of the NGT 200 may not properly reach the stomach. In one aspect of this disclosure, the back of the throat may be visually inspected for kinks in the NGT 200. In one aspect of this disclosure, the NGT 200 may be measured and marked before the NGT 200 is inserted into the patient. More specifically, measurements may be identified on the NGT 200 that represent the distance from the nose, via the earlobe, to the stomach. The NGT 200 is marked so that during insertion, when the stomach mark of the NGT 200 is at the nose, the distal end and opening 162 of the NGT 200 should be in the stomach.

When the stomach mark of the NGT 200 reaches the nose and the distal end of the NGT 200 reaches the stomach, a decrease in airflow impedance is typically evident. When airflow impedance is perceived as traversing the esophagus and the inserted NGT 200 length mark indicates that the stomach has been reached and yet the pulse duration remains unchanged, this may be an indication of a kink in the NGT 200. More specifically, if the mark on the NGT 200 indicates the opening 162 should be positioned in the stomach, the impedance of the NGT 200 should be relatively reduced. If impedance is not reduced at this length, a kink in the NGT 200 may be causing the higher than expected impedance.

A kinked NGT 200 has a reduced effective length i.e. air volume trapped in the NGT 200 between the location of the kink and the apparatus 202. This reduced air volume will be evident as an increase in measured airflow impedance relative to airflow impedance when the tube is in the esophagus. As such, one aspect of this disclosure considers identifying a kink in the NGT 200 when impedance is higher than expected.

NGTs come from multiple manufacturers in different French sizes (or diameters) and lengths. They are made from many different materials with a variety of characteristics. A brief calibration of each NGT 200 by the apparatus 202 may be executed to compensate for all the potential differences in NGT design. Furthermore, the tolerances of components used in the manufacture of the apparatus 202 can be relaxed since they too will be compensated for by the calibration.

In one method of calibration, the opening 162 of the NGT 200 may be obstructed in what mimics the NGT 200 condition when the opening 162 is positioned in the esophagus. This calibration process may register pump 105 action and duration while the opening 162 of the NGT 200 is blocked. From this pulse duration time, a pulse duration time expected for the trachea and stomach can be derived mathematically. In other words, the expected pulse duration time for the relatively high impedance when the opening 162 is in the esophagus and the comparatively low impedance when the opening 162 is in the trachea can be generated based on the pulse duration time identified when the opening was obstructed.

In another aspect of this disclosure, a ‘Whoosh Test’ may be used to confirm that the opening 162 of the NGT 200 is in the stomach. For a Whoosh Test, a small amount of air is injected into the NGT 200 while auscultating the stomach area by a healthcare professional. A “whoosh” sound identified by the healthcare professional is an indication that the NGT 200 is in the stomach. This method is an effective way to periodically reassure the healthcare professional that the opening 162 of the NGT 200 has not been dislodged from the stomach back up into the esophagus. If no whoosh sound is detected, the NGT 200 may be further inserted into the user until a whoosh sound is detected.

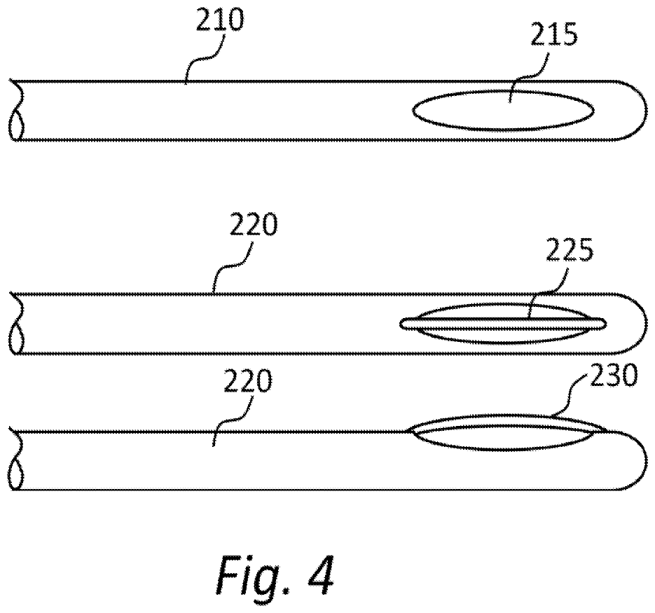

FIG. 4 illustrates examples of other embodiments of the opening 162 contemplated herein. The openings 215 and 225 are illustrated at the distal end of a NGT 210 and 220. The eye-like opening 225 shows an optional protective member 230 against obstruction. Alternately, two eye-like openings on opposite sides of the distal tube wall will avoid such obstruction. However, other embodiments may have four lateral eye-like openings distributed around the NGT 200. This feature enables the NGT 200 to be exposed to air when in the trachea even in the presence of an endotracheal tube or the like. More specifically, because of the small available area with which to maneuver, a definite determination of location of the opening 215 ahead of reaching the endotracheal cuff may not be possible. However, by the time the opening 215 of the NGT 200 has bypassed the cuff and before reaching the lungs, it will be exposed to air, and lower the airflow impedance in the NGT 200 will be identifiable utilizing the apparatus 202 discussed herein.

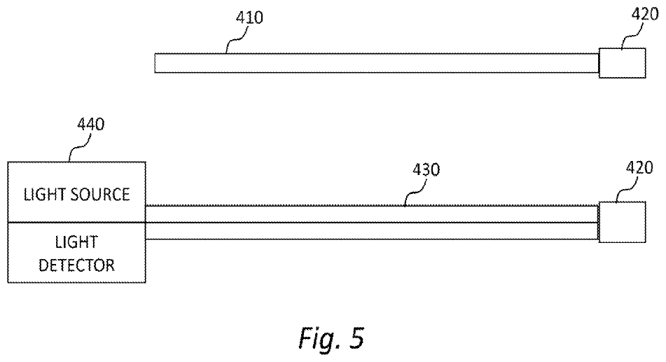

FIG. 5 illustrates a thin probe 410 that may be about 0.5 mm in diameter 410 in one example. The lengths of the probe 410 can exceed the length of the NGT 200. A tip 420 of the probe 410 is coated with a thin layer of a chemical, such as, but not limited to, Magnesium Oxide (“MgO”) or Tin Oxide (“SnO”) that changes color as a result of a reaction with gastric fluids such as hydrogen chloride (“HCl”). When the probe is removed from the lumen of the tube, a visual inspection is performed to ascertain if color change occurred. If the tip 420 changed color, it is an indication that it was in contact with gastric fluids through the opening 162 on the distal end of the NGT 200. This confirms that the opening 162 of the NGT 200 reached the stomach regardless of the pH level of the stomach content.

An alternative method for verification that the opening 162 of the NGT 200 has reached the stomach is an optical sensor that can be integrated with the apparatus 202. A two strand fiber optic bundle 430 may be coated with a thin layer of a chemical, such as, but not limited to, MgO or SnO 420 that changes color as a result of a reaction with gastric fluids such as HCl. The other end of the optical fibers is attached to an optical transceiver 440 that includes a light source and light sensor. Before the reaction with HCl acid, both MgO and SnO are white and the intensity of the reflected light is high. After the encounter with HCl in the stomach, MgCl2 is transparent and SnCl2 is black, the intensity of the reflected light is low. Other nontoxic chemicals with similar properties can achieve the same effect.

Both the manual probe 410 and the fiber-optic probe 430 must have chemical 420 securely adhered and any cement used must be porous so that acid can reach the chemicals 420 respectively and show as transparent or translucent and the color of the chemical 420 can be observed.

In case the stomach is full from a previous feeding and indicates a neutral pH level, the air flow impedance measurement will indicate lower impedance than in the esophagus. Optionally, verification can be done after the stomach content is digested. Yet another option is visual verification by aspiration of gastric content and detection of acidity, color etc.

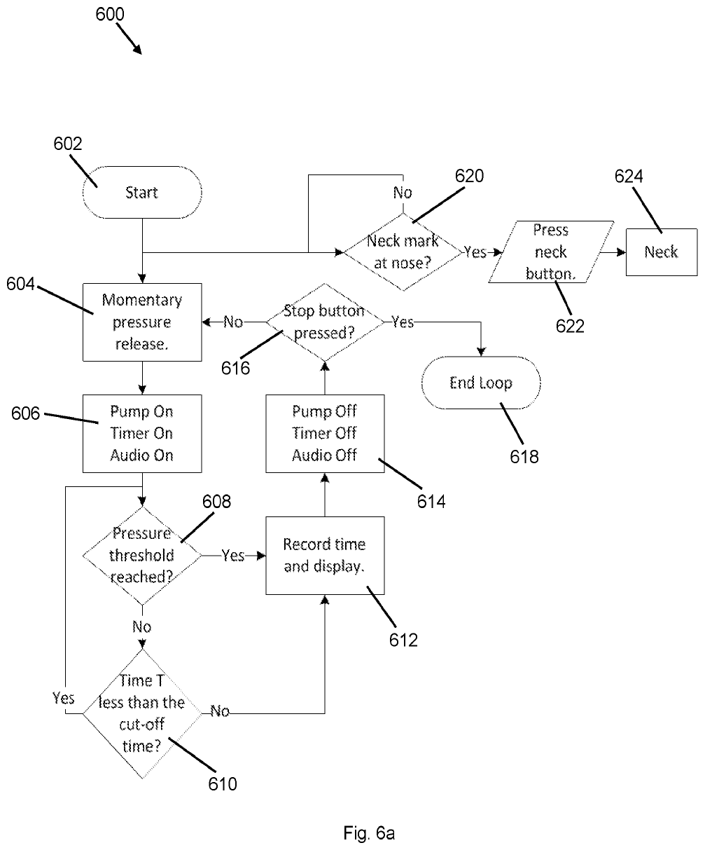

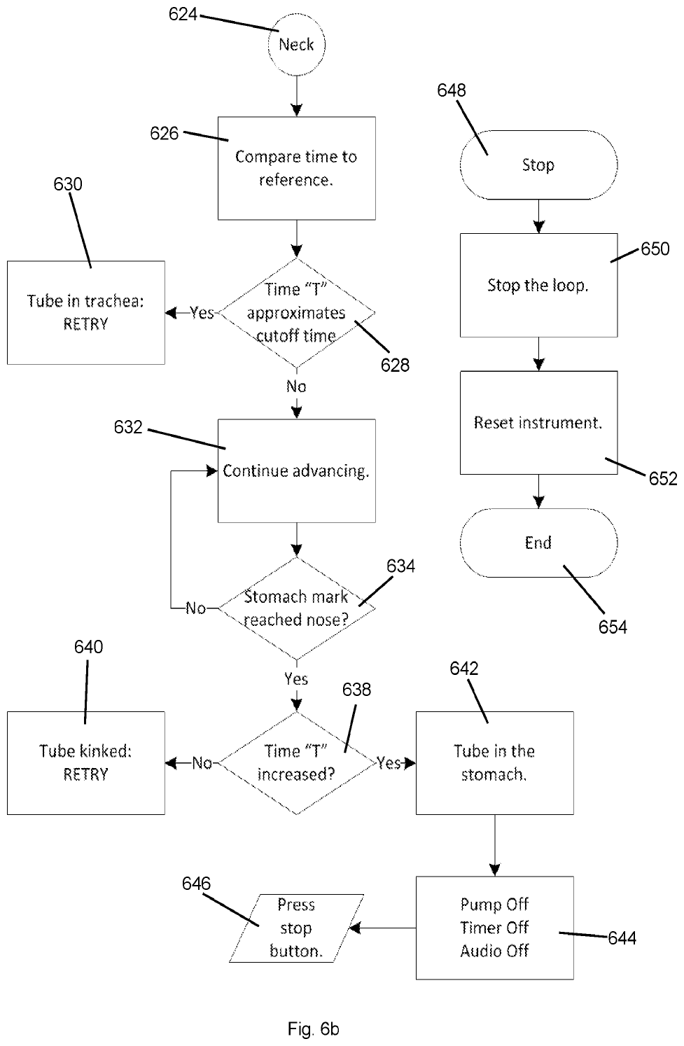

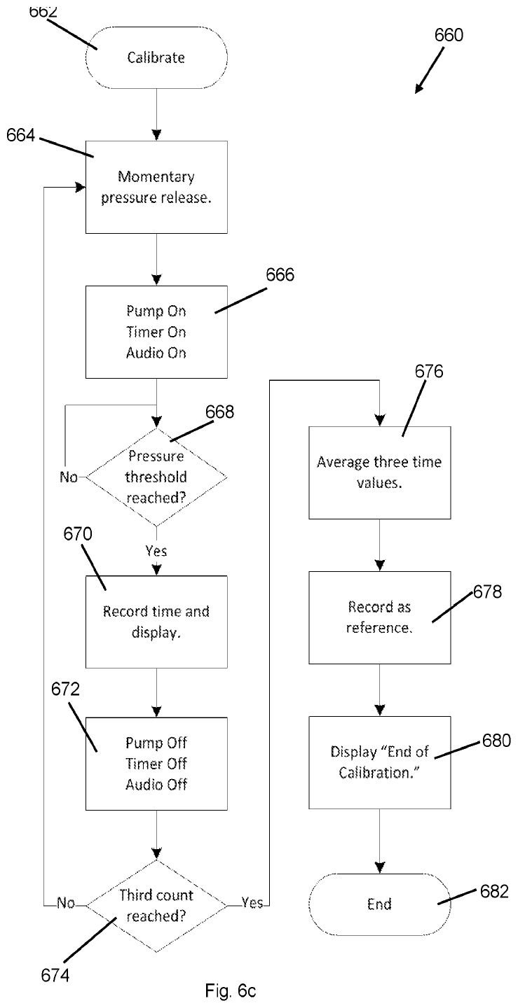

Referring now to FIGS. 6a-6c, one embodiment of a logic flow chart 600 for implementing the present disclosure is illustrated. In this embodiment, the apparatus 202 may be initiated at a power on or other start function illustrated in box 602. The apparatus 202 may be coupled to the NGT 200 to utilize pressure variances and fluid flow to determine the location of the opening 162 at the distal end of the NGT 200 as discussed herein. After the start at 602, the apparatus 202 may momentarily release any pressure in the NGT 200 or otherwise built up in the apparatus 202 in box 604. The pressure release of box 604 may ensure that the pressure within the NGT 200 is equalized to the atmospheric pressure. As discussed herein, one aspect of this disclosure considers the relative pressure change within the NGT 200 after the pump 105 has processed a volume of fluid to, or from, the NGT 200. Accordingly, the pressure release of box 604 may ensure the pressure within the NGT 200 is equalized to the surrounding atmosphere prior to advancing.

While an explicit pressure release function is discussed herein, other embodiments may not implement a pressure release function but rather assume the NGT 200 has an interior pressure equalized to the environment 158 prior to executing the logic flow chart 600.

The pump 105, timer, and audio/video component such as the display 140 may be powered on in box 606. This may include engaging the pump 105 with the pump control 125 to pump a volume of fluid into, or out of, the inner cavity 154 of the NGT 200 coupled to the apparatus 202. At substantially the same time, a timer may be started to identify the length of time the pump 105 has been actively pumping. As the pump 105 is pumping fluid to, or from, the NGT 200, the pressure indicated by the pressure sensor 110 may be compared to a pressure threshold in box 608.

At box 608, the measured pressure of the NGT 200 may be compared to a pre-determined pressure threshold. The predetermined pressure threshold may be a typical pressure produced by the pump 105 when the distal end of the NGT 200 is at least partially blocked. The predefined or predetermined pressure threshold may be the minimum threshold pressure that is sufficiently high to effectively execute the logic flow chart 600. In one example, the predefined pressure threshold may be around 5 mmHg. However, in other examples considered herein the predefined pressure threshold may be more than, or less than 5 mmHG and may vary slightly depending on the specific application. In one aspect of this disclosure, the predefined threshold pressure may be as low as possible to minimize the impact on soft tissue that may be surrounding the opening 162 as fluid is pumped into, or out of, the inner cavity 154.

If the measured pressure is not greater than the pressure threshold in box 608, the timer may be considered to determine whether the pump 105 has been operating for less time than a cut-off time threshold in box 610. If the pump 105 has only been operating for an amount of time that is less than the cut-off time threshold, the pump 105 may continue to run and the pressure monitored in box 608. However, if the cut-off time is greater than the cut-off time threshold in box 610, or the pressure is threshold is reached in box 608, the recorded time of the timer may be displayed on the timer display 140 in box 612. In box 614, the pump 105, timer, and audio may be turned off. In box 616, a stop button may be monitored. If the stop button was engaged, apparatus 202 may end the logic flow chart 600. However, if the stop button has not been engaged in box 616, the logic flow chart 600 may execute a momentary pressure release of box 604 as discussed herein and continued through boxes 606, 608, 610, 612, 614, and 616.

In yet another aspect of the logic flow chart 600, the apparatus 202 may have an input to allow a user to identify when a neck mark on the NGT 200 is positioned at the nose. As discussed herein, a neck mark may be created on the NGT 200 at a location that is representative of the length the NGT 200 should be inserted through the nose before entering the esophagus. As such, box 620 may be directed towards identifying when the neck mark is at the nose. If the neck mark is identified at the nose at 620, a neck button on the apparatus 202 may be selected by the user at 622 indicating the positioning of the NGT 200. When the neck button is selected in box 622, a neck function 624 may be implemented.

The neck function 624 is illustrated in more detail in FIG. 6b. More specifically, the neck function 624 may determine whether the elapsed time is at about the cut-off threshold in box 626. If the elapsed time is at about the cut-off threshold in box 628, that indicates that the fluid being pumped into, or drawn from, the NGT 200 was not substantially impeded at the distal end. Accordingly, if the pressure of the fluid in the NGT 200 has not reached the threshold pressure within about the cut-off threshold time in box 628 and the neck mark is at the nose, it may be an indication that the opening 162 at the distal end of the NGT 200 is in the trachea rather than the esophagus. As such, box 630 may provide an indication of such on a display, audibly, or using any other known indicator.

However, if the pressure in the NGT 200 does reach the pressure threshold at or before the cut-off threshold time, it may be an indication that the opening 162 in the distal end is properly positioned in the esophagus. As such, the user may continue advancing the NGT 200 down the esophagus towards the stomach in box 632. Further, a stomach mark may be positioned on the NGT 200 indicating when the NGT 200 is likely positioned in the stomach as discussed herein. As such, the user may continue advance the NGT 200 into the patient until the stomach mark is at about the nose in box 634. At this point, the distal opening at the end of the NGT 200 should be positioned within the stomach. Accordingly, in box 638 the pressure in the NGT 200 may be monitored to determine whether the opening in the distal end of the NGT 200 is in the stomach. More specifically, if it does not take longer for the pump 105 to generate the threshold pressure in the NGT 200 in box 638, it may be an indication that the NGT 200 is kinked at box 640. At this time, the apparatus 202 may provide a warning or notice regarding the kink.

However, if in box 638 the time is increased to pressurize the NGT 200, it may be an indication that the opening at the distal end of the NGT 200 is positioned in the stomach and not substantially impeded. As such, in box 642 it may be determined that the opening at the distal end of the NGT 200 is in the stomach and an indication may be provided by the apparatus 202 identifying the same. Once the distal end is identified as in the stomach, the pump 105, timer, and any audio or visual indicators may be powered off in box 644. The user may also select a stop button at 646 to end the neck function 624.

The apparatus 202 may also have a stop function 648 that can be selectively engaged by the user or controller. The stop function 648 may automatically end any logic loops currently being implemented by the apparatus 202 in box 650. Further, the stop function 648 may also reset all values and thresholds in box 652. Lastly, the stop function 648 may end at 654 and the apparatus 202 may be ready for subsequent use.

Referring now to FIG. 6c, the logic flow for a calibration process 660 is illustrated. The calibration process 660 may be implemented to identify the time it takes for the interior region 154 of the NGT 200 to reach a predefined pressure when the opening 154 on the distal end of the NGT 200 is substantially blocked. In one aspect of this disclosure, different types of NGT 200 may be coupled to the apparatus 202. The different types of NGT 200 may have different inner volumes, lengths, and rigidity among other things. Accordingly, the calibration process 660 may be implemented to establish expected time thresholds for the NGT 200 to meet the expected pressure levels.

In one aspect of this disclosure, the pump 105 may pump at a substantially consistent flow rate. As such, the amount of time it may take to generate the pressure threshold within the NGT 200 depends, at least in part, on the volume of the inner chamber of the NGT 200. The calibration process 660 discussed herein allows the apparatus 202 to establish the particular time threshold for the particular NGT 200 coupled thereto by determining the amount of time it takes to get the inner chamber of the NGT 200 to the pressure threshold based on a substantially fixed flow rate from the pump 105.

More specifically, in box 662 the user may select a calibrate button. Alternatively, in one embodiment the calibration process 660 may be automatically implemented by the apparatus 202 when a new NGT 200 is coupled thereto. Regardless, the calibration process 660 should be implemented when an NGT 200 is fluidly coupled to the apparatus 202 and the opening or openings at the distal end of the NGT 200 are substantially blocked. Once the NGT 200 is coupled to the apparatus 202 and the distal end is substantially blocked, a pressure release function may be implemented in box 664. The pressure release function of box 664 may utilize the pressure release 115 of the apparatus 202 discussed herein to equalize the pressure within the inner chamber 154 of the NGT 200 with the surrounding atmosphere 158. More specifically, the pressure release 115 may be a valve between the inner chamber of the NGT 200 and the surrounding atmosphere 158 that temporarily opens in box 664.

After the momentary pressure release, the pressure release 115 may close, substantially isolating the inner chamber 154 of the NGT 200 from the surrounding atmosphere 158. Once the pressure release 115 is closed, the pump 105 and timer may be initiated in box 666. The timer may be an internal timer on a controller of the apparatus 202 or identified from a separate timing component. Regardless, the timer may record the amount of time the pump 105 is engaged or otherwise powered. Further, in one aspect of this disclosure an audio or visual indicator may be initiated when the pump 105 is engaged in box 666.

Once the pump 105 is powered in box 666, the pressure sensor 110 may be monitored to determine when the pressure of the inner cavity of the NGT 200 reaches a predefined pressure threshold in box 668. The pressure sensor 110 may be fluidly coupled to the inner chamber 154 of the NGT 200 to identify the pressure therein. Further, the predefined pressure threshold may be a pressure that is greater, or less than, the surrounding atmospheric pressure. In other words, the pump 105 may be pumping fluid into, or out of, the inner chamber 154 of the NGT 200. If the pressure identified by the pressure sensor 110 is not within the predefined pressure threshold, the pump 105 may continue to pump fluid to, or from, the inner cavity of the NGT 200. However, once the pressure sensor 110 identifies a pressure that is within the predefined pressure threshold, the time it took for the pump 105 to generate a pressure in the inner chamber of the NGT 200 is recorded as data and displayed on the timer display 140 in box 670.

After the time it took the pump 105 to generate a pressure in the NGT 200 within the predefined pressure threshold is established, the pump 105 and timer may be powered off in box 672. The calibration process 660 may then determine whether the number of time readings reaches a predefined count in box 674. In other words, the calibration process 660 may execute boxes 664, 666, 668, 670, and 672 a number of times in order to identify an average time it takes the pump 105 to generate a pressure within the pressure threshold for the NGT 200. Accordingly, in box 674 if the number of times recorded does not meet the predetermined count, boxes 664, 666, 668, 670, and 672 will be repeated. In the non-exclusive example of FIG. 6c, three separate time readings may be recorded before advancing to box 676. However, other embodiments consider doing fewer or more time readings as well.

Once the predefined number of counts is reached in box 674, the average time it took the pump 105 to generate the predefined pressure will be determined in box 676. This may include determining an average time based on all of the recorded times in the data. In box 678, once the average time is calculated in box 676, the average time may be saved in the data as a reference for the time threshold values used in boxes 610 and 628 among others. In box 680 the display 140 or other visual or audio device may indicate that the calibration process is completed. Once the user has been notified of the completion of the calibration process in box 680, the calibration process 660 may be ended in box 682.

The logic discussed herein for FIGS. 6a-6c may be executed by one or more controller that is electronically coupled to, or otherwise in communication with, the apparatus 202. The controller or controllers may include a processor for executing commands and processing data among other things. Further, the controller or controllers may have, or have access to, a memory unit that can store data. In one example, a controller is electronically coupled to the apparatus 202. The pump 105 is selectively powered by the controller based on the logic discussed herein (i.e., the controller initiates the pump control 125). Further, the controller is in communication with the pressure sensor 110 to identify the pressure of the inner chamber 154 of the NGT 200 when fluidly coupled thereto. The controller may also selectively control the position of the pressure release 115. The controller of the apparatus 202 may also receive user inputs from any input device such as the test trigger 130 and display or otherwise generate indicator to the user through the timer display 140 or other audio or visual component that can be controlled, in part, by the controller.

The pump 105 is discussed herein as pumping fluid into, or out of, the NGT 200. The term “fluid” may refer to a gas or liquid state or combination thereof. In one example, the pump 105 pumps gas from the surrounding atmosphere into the NGT 200. Alternatively, the pump 105 may pump gas out of the NGT 200. Further still, the pump 105 may be a controlled source of air flow such as a compressed air reservoir or the like for providing air into, or out of, the NGT 200.

While a particular apparatus and method are discussed herein, other devices and methods can be utilized to implement the teachings of this disclosure. More specifically, any device capable of moving fluid into, or out of, an NGT may be used to implement these teachings. The time it takes the device to reach a predetermined pressure may be monitored to identify impedance at an opening of the NGT. Alternatively, the volume of fluid removed from, or added to, the NGT before the predetermined pressure is achieved may be monitored to identified the location of the opening of the NGT.

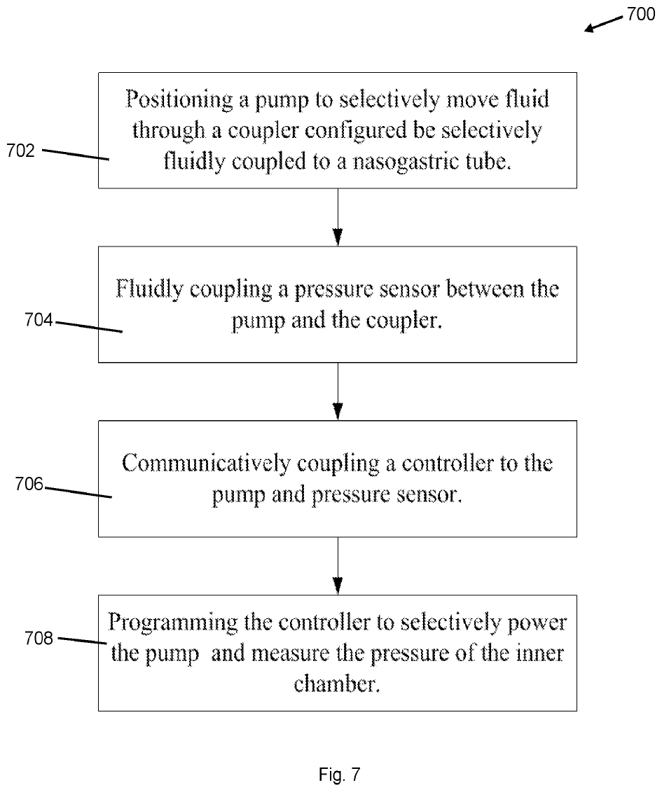

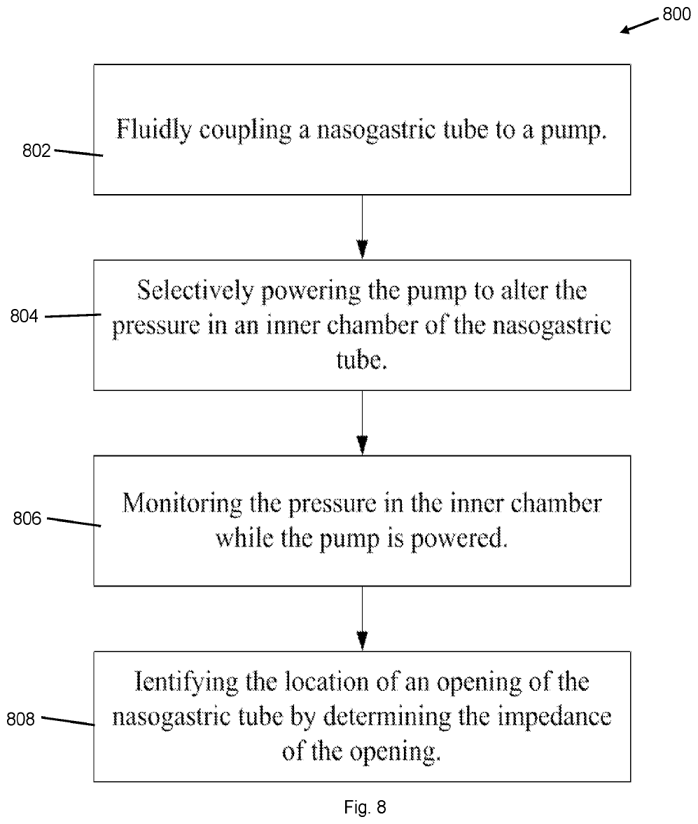

Referring now to FIG. 7, one example of a method of manufacturing a device 700 of this disclosure is illustrated. The method of manufacturing 700 includes positioning a pump to selectively move fluid through a coupler configured be selectively fluidly coupled to a nasogastric tube in box 702. The pump may be pump 105 and the coupler may be coupler 145 or 150 depending on whether a filter is included. The pump 105 is positioned to be fluidly coupled to an NGT when coupled to the coupler to selectively move fluid to or from the inner chamber 154 of the nasogastric tube 200 when fluidly coupled thereto.

The manufacturing method 700 may include fluidly coupling a pressure sensor between the pump and the coupler in box 704 to selectively identify a pressure of the inner chamber 154 when coupled to the nasogastric tube 200. The pressure sensor may be the pressure sensor 110 and may be fluidly coupled to any portion of conduit or the like between the pump and the NGT 200.

In box 706, a controller may be communicatively coupled to the pump and pressure sensor to provide instructions to selectively power the pump and selectively identify the pressure of the inner chamber. The controller may be controller 166 and may have a wired connection to selectively power the pump or a pump controller. Alternatively, the controller may wirelessly communicate with the pump or pump controller to selectively power the pump. Similarly, the controller may communicate with the pressure sensor through a wired or wireless communication protocol.

In box 708, the controller may be programmed to selectively power the pump for a predetermined amount of time and measure the pressure of the inner chamber when fluidly coupled to the nasogastric tube to identify an impedance of an opening of the nasogastric tube utilizing the teachings considered herein. In one embodiment, the controller is programmed to discontinue powering the pump if the pressure of the inner chamber does not meet a pressure threshold after a predetermined amount of time powering the pump as discussed herein. Further, the controller may have a calibration process programmed therein to establish a time threshold for a specific nasogastric tube. In this embodiment, the controller may store the time threshold established during the calibration and use the time threshold as the predetermined amount of time.

Part of the manufacturing method 700 may include providing a user input in communication with the controller. The user input may be selectable by a user when the nasogastric tube is at a predefined location in a patient. The controller may further be programmed to power the pump and monitor the pressure sensor when the user input is engaged. The manufacturing method may also include programming the controller to communicate with a stop button wherein the controller is configured to not power the pump when an input from the stop button is identified.

Referring now to FIG. 8, a method of identifying the location of a NGT 800 utilizing the teachings of this disclosure is provided. More specifically, the method includes fluidly coupling a nasogastric tube to a pump 802 such as the pump 105 discussed herein. The pump may then be selectively powered to alter the pressure in an inner chamber of the nasogastric tube 804. As the pump is powered, the pressure in the inner chamber may be monitored 806. The information regarding the duration of time the pump was powered and the pressure of the inner chamber may be considered to identify the location of an opening of the nasogastric tube 808. More specifically, the pump duration and pressure may be utilized to determine the impedance of the opening.

As part of this method 800, the impedance may be determined by monitoring the time interval that the pump is powered and the pressure in the inner chamber. Further, this method 800 may determine the impedance at least twice while inserting the nasogastric tube into the stomach of the patient.

While an exemplary embodiment incorporating the principles of the present application has been disclosed hereinabove, the present application is not limited to the disclosed embodiments. Instead, this application is intended to cover any variations, uses, or adaptations of the application using its general principles. Further, this application is intended to cover such departures from the present disclosure as come within known or customary practice in the art to which this present application pertains and which fall within the limits of the appended claims.

1. An apparatus for identifying the location of a nasogastric tube, comprising:
a pump configured to be selectively fluidly coupled to a nasogastric tube;
a pressure sensor configured to identify the pressure of an inner chamber of the nasogastric tube; and
a controller that selectively powers the pump and reads the pressure sensor to identify an impedance of at least one opening of the nasogastric tube.

2. The apparatus of claim 1, further comprising a filter positioned to filter fluid passing between the pump and the nasogastric tube.

3. The apparatus of claim 1, further comprising a pressure release configured to selectively fluidly couple the inner chamber of the nasogastric tube to a surrounding environment.

4. The apparatus of claim 1, wherein the controller has a pressure threshold stored therein.

5. The apparatus of claim 4, wherein the controller has a calibration process stored therein and the controller selectively executes the calibration process to determine a time threshold for generating the pressure threshold.

6. The apparatus of claim 1, wherein the pump runs intermittently.

7. The apparatus of claim 1, wherein the controller is configured to determine airflow impedance based on a pressure time factor.

8. The apparatus of claim 1, wherein the controller is configured to stop the pump when the pressure sensor identifies that a predetermined pressure is reached.

9. The apparatus of claim 1, wherein the controller is configured to determine the air flow impedance based on the time it takes the pump to generate a threshold pressure in the inner chamber.

10. A method for manufacturing an apparatus for identifying the location of a nasogastric tube, comprising:
positioning a pump to selectively move fluid through a coupler configured be selectively fluidly coupled to a nasogastric tube, the pump configured to selectively move fluid to or from an inner chamber of the nasogastric tube when fluidly coupled thereto;
fluidly coupling a pressure sensor between the pump and the coupler to selectively identify a pressure of the inner chamber when coupled to the nasogastric tube;
communicatively coupling a controller to the pump and pressure sensor to provide instructions to selectively power the pump and selectively identify the pressure of the inner chamber;
programming the controller to selectively power the pump and measure the pressure of the inner chamber when fluidly coupled to the nasogastric tube to identify an impedance of an opening of the nasogastric tube.

11. The method of claim 10, further comprising programming the controller to discontinue powering the pump if the pressure of the inner chamber does not meet a pressure threshold after a predetermined amount of time powering the pump.

12. The method of claim 11, further comprising programming a calibration process in the controller to establish a time threshold for a specific nasogastric tube.

13. The method of claim 12, further comprising programming the controller to store the time threshold established during the calibration and use the time threshold as the predetermined amount of time.

14. The method of claim 10, further comprising providing a user input in communication with the controller and configured to be engaged by the user when the nasogastric tube is at a predefined location in a patient.

15. The method of claim 1514, further comprising programming the controller to power the pump and monitor the pressure sensor when the user input is engaged.

16. The method of claim 10, further comprising programming the controller to communicate with a stop button wherein the controller is configured to not power the pump when an input from the stop button is identified.

17. A method of identifying the positioning of a nasogastric tube in the stomach of a patient, comprising:
fluidly coupling a nasogastric tube to a pump;
selectively powering the pump to alter the pressure in an inner chamber of the nasogastric tube;
monitoring the pressure in the inner chamber while the pump is powered; and
identifying the location of an opening of the nasogastric tube by determining the impedance of the opening.

18. The method of claim 17, wherein the impedance is determined by monitoring the time interval that the pump is powered and the pressure in the inner chamber.

19. The method of claim 17, wherein the impedance is determined at least twice while inserting the nasogastric tube into the stomach of the patient.
